(19) United States
(12) Patent Application Publication
(10) Pub. No.: US 2026/0076888 A1
(43) Pub. Date: Mar. 19, 2026

(54) Skincare Compositions For Controlled Delivery of Active Agents

(71) Applicant: ELC Management LLC, Melville, NY (US)

(72) Inventors: Milan Franz Sojka, Algonquin, IL (US); Joseph Mirador, Westbury, NY (US); Wilson A. Lee, Hauppauge, NY (US)

(21) Appl. No.: 18/886,117

(22) Filed: Sep. 16, 2024

(51) Int. Cl. A61K 8/81; A61K 8/34; A61K 8/73; A61Q 19/00

(52) U.S. Cl. CPC A61K 8/8152; A61K 8/347; A61K 8/731; A61Q 19/00; A61K 2800/95

(57) ABSTRACT

An aqueous topical skincare composition that comprises one or more skin active agents, such as bakuchiol; microcrystalline cellulose; and a film forming polymer blend, wherein the polymer blend is a combination of acrylates copolymer and acrylates/vinyl acetate copolymer.

### FIELD OF THE INVENTION

The present invention is in the field of topical preparations for keratinic surfaces. More specifically, the invention pertains to safe, stable film-forming compositions that are able to efficiently deliver active agents into the skin, in a controlled manner.

### BACKGROUND OF THE INVENTION

The present invention is concerned with percutaneous absorption of active agents. Percutaneous absorption is a diffusion process that depends on size of the contact area between the a permeant and the skin surface. The size of the contact area is heavily influenced by skin wettability, which, in turn, depends on the surface tension of the wetting agent. Critical surface tension (CST), a property of the material being wetted, is the minimal surface tension that a liquid must have to spread over the surface of a substrate to give complete wetting. When the surface tension of the liquid is less than CST, the liquid will more readily spread over the surface. When the surface tension of the liquid is greater than CST, the liquid will wet out less well, or tend to form droplets. In the case of a liquid (such as, water) at rest on a solid surface, it is well known that the angle of contact between the liquid and solid surface is a reliable indicator of wetting. When the contact angle is less than 90° the liquid tends to spread, or wet, the solid surface. When the contact angle is greater than 90° the liquid tends bead on the solid surface. In scientific literature, the critical surface tension of human skin and the water contact angle have been reported for certain locations of the body (usually, the hand, forearm or forehead), and for various pretreatments of the skin (see for example, F. Eudier, et al., “Skin Surface Physico-chemistry: Characteristics, methods of measurement, Influencing factors and future developments,” Advances in Colloid ad Interface Science 264 (2019) 23-26). In general, the presence of sebum on the skin has a significant impact on critical surface tension and wettability. For the forearm, an area of low sebum, critical surface tension values between 22.2 and 30.6 mN/m have been reported. For the forehead, an area of relatively higher sebum concentration, a critical surface tension value of 50.7 mN/m has been reported.

In the present context, measurements on the forehead are of particular interest. The forehead skin, with no pretreatment cleansing, is conclusively shown to be hydrophilic (i.e. having contact angles less than 90°). When pretreatment, such as cleansing, removes a substantial amount of sebum, the treated forehead skin tends toward hydrophobicity. Overall, the presence of sebum on skin tends to lower hydrophobicity, decrease the water contact angle, increase surface free energy and critical surface tension, and improve wettability, leading to better percutaneous absorption of active agents in a composition applied to the skin.

Bakuchiol is a meroterpenoid compound. The preferred IUPAC designation is 4-[(1E,3S)-3-Ethenyl-3,7-dimethylocta-1,6-dien-1-yl]phenol (C₁₈H₂₄O; CAS #10309-37-2). It is commonly obtained as an extract of the seeds of the Psoralea corylifolia plant (also known as Cullen corylifolium). In the cosmetic and personal care field, bakuchiol has been promoted as an alternative to retinol for treating photodamage, such as wrinkles and hyperpigmentation. Some evidence exists to suggest that bakuchiol is as effective as retinol, but with fewer side effects, which usually include peeling, stinging and redness. Nevertheless, bakuchiol is not without some side effects which must be considered when developing a topical skin care product. The challenge is to control the rate of penetration into the skin, so that effective tissue resolution and cell rejuvenation may occur without adverse effects, such as irritation and redness.

In the following co-owned applications, U.S. Ser. No. 15/632,903 (now U.S. Pat. No. 11,103,439), U.S. Ser. No. 15/906,372 (now U.S. Pat. No. 10,813,874), U.S. Ser. No. 17/029,147 (now U.S. Pat. No. 11,434,399), U.S. Ser. No. 17/176,527, U.S. Ser. No. 16/816,995 (now U.S. Pat. No. 11,291,618), U.S. Ser. No. 16/197,858, U.S. Ser. No. 16/381,806 (now U.S. Pat. No. 10,507,175), U.S. Ser. No. 16/267,441 (now U.S. Pat. No. 10,980,717), U.S. Ser. No. 17/189,768 (abandoned), U.S. Ser. No. 16/827,876 (now U.S. Pat. No. 11,129,788), and U.S. Ser. No. 18/168,776 the applicant has disclosed specific combinations of acrylates/vinyl acetate copolymer and acrylates copolymer in an aqueous base, and various useful properties of these combinations that depend on the intended application and the presence of certain other ingredients. Upon application to a skin surface, these compositions, although water based, dry to a film that is clear, flexible and comfortable. Co-owned application, U.S. Ser. No. 17/806,294, discloses topical compositions for controlled release of active ingredients that comprise 20% to 60%, by weight, of a combination of acrylates copolymer and acrylates/vinyl acetate copolymer, wherein the weight ratio of acrylates/vinyl acetate copolymer to acrylates copolymer ranges from 10:1 to 30:1, preferably 15:1 to 25:1, more preferably about 20:1. The compositions also comprise 5.5%-10%, preferably 6%-7% of a combination of propane-1,3 diol and glycerin, which can be used to fine tune the pore size, as needed. The weight ratio of propane-1,3 diol to glycerin ranges from about 1:1 to about 4:1. It is disclosed that when these compositions are applied to a substrate, such as a keratinic surface, and allowed to dry, the dried film will have an average porosity between about 0.1 μm to and 10 μm.

Microcrystalline cellulose (MCC) is a known additive for cosmetic and personal care products, where it has various uses related to product stability and aesthetics. MCC is prepared using naturally occurring cellulosic materials as a precursor. The cellulosic precursor material is processed to organize the material into microfibrils, which are formed from partially polymerized glucose monomers. A high-degree of internal bonding between fibrils gives the cellulose precursor material a crystalline structure, albeit with amorphous regions. These amorphous regions may be removed by various treatments to produce microcrystalline cellulose. MCC is characterized by a large surface area and high internal porosity, which gives MCC a high water retention capability. Reported uses of microcrystalline cellulose include as an abrasive, absorbent, anti-caking agent, bulking agent, emulsion stabilizer, slip modifier and viscosity increasing agent.

To the best of our knowledge, the use of acrylates/vinyl acetate copolymer, acrylates copolymer and microcrystalline cellulose in a topical composition for the purpose of regulating the delivery of active ingredients, such as bakuchiol, into the skin is unknown. There remains a need for a topical skin care composition that offers the benefits of bakuchiol and other potentially irritating active ingredients without the negative side effects.

### OBJECT OF THE INVENTION

A main object of the invention is to provide a delivery system for controlled release of active agents into the skin.

Another object of the invention is to provide a delivery system for controlled release of bakuchiol into the skin.

### SUMMARY OF THE INVENTION

The foregoing objective is achieved in a topical skincare composition that comprises (a) one or more skin active agents, such as bakuchiol; (b) 20% to 60% (by weight) of a polymer blend, wherein the polymer blend comprises a combination of acrylates copolymer and acrylates/vinyl acetate copolymer, wherein the weight ratio of acrylates/vinyl acetate copolymer to acrylates copolymer ranges from 10:1 to 30:1, preferably 15:1 to 25:1, more preferably about 20:1; and (c) 0.5% to 5% of microcrystalline cellulose in an aqueous mixture.

### DETAILED DESCRIPTION

Topical skincare compositions of the present invention take a two-step approach to delivery of active agents. Separately addressed are the rate at which the active agent is presented at the skin surface, as well as the rate at which the active agent is absorbed through the stratum corneum.

Compositions according to the present invention are aqueous, and comprise a film-forming polymer blend, microcrystalline cellulose, and an active agent, preferably a hydrophobic active agent, for delivery into the skin. The compositions of the invention are wet when applied to the skin. In the wet state, the film-forming polymer blend is hydrophilic. After application to the skin, the polymer blend dries to form a porous hydrophobic film. As the polymer blend transitions to hydrophobic, the aqueous component of the composition (which includes the MCC) tends to accumulate below the porous polymer film. This happens, at least in part, because the aqueous mixture of MCC has a higher specific gravity (about 1.07-1.15) than the film forming polymer blend (about 1.02-1.05). As a result, when the polymer blend has fully dried, the microcrystalline cellulose is trapped below the film, where it forms a matrix of hydrophilic fibers adjacent to the skin surface.

Once some amount of the composition has been applied to the skin, and allowed to set up, the dried polymer film traps the microcrystalline cellulose and other aqueous components in a volume of space beneath the film. Over time, water moves in and out of the volume, through the stratum corneum. The water may come from the composition, from sweating or from normal transepidermal water loss (TEWL) that occurs even when the body is at rest. As a matter of course, the temperature of the skin rises and falls throughout the day depending on one's level of activity, and on the temperature of ambient environment. As temperature rises, the volume of space beneath the polymer film expands and more water from inside the body enters that space. As skin temperature drops the volume shrinks, which causes pressure to build up within the volume, and causing the skin to reabsorb some of the water. Throughout the time in which the composition remains on the skin, the rise and fall of temperature causes the pressure within the volume to fluctuate. This fluctuation creates a pumping action with the net effect of driving solute (the active agent) toward and into the skin surface. Features of the invention that aid the penetration of active agent into the epidermis will be discussed below.

### Water

Compositions of the invention are aqueous, and typically comprise about 25%-75% of water by weight of the total composition. Preferably, the total amount of water is at least 50%, and more preferably, at least 60%. This amount of water is that from all sources, such as that in Vinysol 2140L and Daitosol 5000AD (see below).

### Film-Forming Polymer Blend

The film-forming polymer blend comprises acrylates/vinyl acetate copolymer and acrylates copolymer. Acrylates/vinyl acetate copolymer (C₁₅H₂₆O₄; also known as ethenyl acetate; 2-ethylhexyl prop-2-enoate; CAS number 25067-02-1; for detailed information, see PubChem Compound Database; CID=168269) is a tacky material, with the following monomer structures.

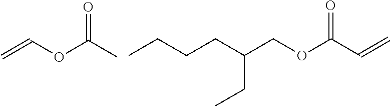

In cosmetics, this tacky material often functions as a binder, film former, adhesive and/or hair fixative. When deployed in aqueous cosmetic systems, acrylates/vinyl acetate copolymer can impart a film on the skin or hair. Acrylates/vinyl acetate copolymer is commercially available, for example, as Vinysol 2140L from Daido Chemical Corp. Vinysol 2140L is a 46.6% aqueous mixture of acrylates/vinyl acetate copolymer. Vinysol 2140L is reported to have a pH of 4.5, a viscosity of 2,000 mPa-s, a calculated glass transition temperature (T_{g}) of −9° C., while the film exhibits a break elongation of 1,200%, and a break strength of 1.2 MPa (when spread to a thickness 0.1 mm). By itself, the acrylates/vinyl acetate copolymer is somewhat too rigid for consumer acceptance.

To address the problem of high rigidity, the acrylates/vinyl acetate copolymer was combined with acrylates copolymer (C₁₄H₂₂O₆, also known as ethyl prop-2-enoate; methyl 2-methylprop-2-enoate or 2-methylprop-2-enoic acid; CAS number 25133-97-5; for detailed information, see PubChem Compound Database; CID=168299). This acrylates copolymer has a lower T_{g} than acrylates/vinyl acetate copolymer. In various types of cosmetic formulations, acrylates copolymer has a wide variety of uses including as film formers, hair fixatives, binders, and suspending agents, viscosity enhancers, antistatic agents and adhesives. Acrylates copolymer is commercially available, for example, as Daitosol 5000AD from Daito Kasei Kogyo Co. Daitosol 5000AD is a 50% aqueous mixture of acrylates copolymer. Daitosol 5000AD is reported to have a pH of 5.5-7.5, a viscosity of 50-100 mPa-s, a glass transition temperature (Tg) of about −14° C.

In compositions according to the present invention, the combination of acrylates/vinyl acetate copolymer and acrylates copolymer accounts for 4% to 20% (by weight) of the total composition. The weight ratio of acrylates/vinyl acetate copolymer to acrylates copolymer ranges from 10:1 to 30:1, preferably 15:1 to 25:1, more preferably about 20:1, such as 18:1 to 22:1. When these compositions are applied to a substrate, such as a keratinic surface, and allowed to dry, the dried film develops pores that are useful for holding active agents, and releasing them over time, within the context of the present invention.

The size of the pores is typically between about 0.1 μm to and 10 μm, but, as disclosed in our application U.S. Ser. No. 17/806,294, the size of these pores may be adjusted using propane-1,3 diol and/or glycerin, as needed. As the film dries, some, not all, of the active agent molecules accumulate in the pores of the dried film. In practice, the size of the pores in the dried film will be chosen based on the molecular weight of the active material. In the case of bakuchiol, the average pore size of the film should be about 1 μm to 5 μm. With this average pore size, as the composition dries, at least some bakuchiol will accumulate in the pores of the film, and be released over time. Subsequently, as a result of the pumping action described above, active agent molecules are urged toward the skin. Nevertheless, as so far described, too much active agent might reach the skin surface too fast, resulting in irritation of the skin. To slow down the presentation of active agent at the skin surface, we use a matrix of microcrystalline cellulose.

### Microcrystalline Cellulose

As noted above, when applied to the skin, the polymer-blend tends to set up as a dry film on top of the aqueous component which contains the microcrystalline cellulose. In fact, in the present invention, the polymer film of acrylates/vinyl acetate copolymer and acrylates polymer has a specific gravity of about 1.02-1.05, while the aqueous component with microcrystalline cellulose has a specific gravity of about 1.07-1.15. This layering of the polymer film and the aqueous component has been observed to occur as the film dries. As the active agent is urged toward the surface of the skin, it must pass though the MCC matrix. This matrix, then, can be used as a physical barrier to slow the rate at which an active agent, such as bakuchiol, reaches the skin surface. In general, the hydrophilic MCC may slow the migration of hydrophilic actives toward the skin more than hydrophobic actives.

Additionally, by varying the concentration of MCC in the composition, we are able to alter the surface tension of the aqueous component of the composition. In fact, the greater the concentration of MCC in compositions of the invention, the greater will be the surface tension of the aqueous component that is trapped within the volume of space beneath the dried polymer film. Therefore, the concentration of microcrystalline cellulose in a composition of the present invention can be used to control how well the composition wets out on the skin. And that means that MCC can be used to control the rate of permeation through the skin surface, because a greater rate of permeation will occur when the composition wets out efficiently on the skin surface, and a slower rate of permeation will occur when the composition does not wet out efficiently on the skin surface.

The surface tension of the aqueous component of the composition cannot be easily measured, but the effect of MCC on the surface tension of the wet composition can be correlated. In the case where an active agent might be harmful if it penetrates the skin too quickly, it is useful to adjust the concentration of microcrystalline cellulose so that the surface tension of the wet composition is greater than the surface tension of the skin. For example, the surface tension of the wet composition may be 5% to 20% greater than the surface tension of the skin, such as 10% to 15%. The target surface tension of the composition will depend on the type of skin being treated, dry, normal or oily. For example, for dry skin, a product surface tension that is 15% to 20% greater than that of skin may be appropriate, while for oily skin, 5% to 10% may be better.

In practice, compositions of the invention may be part of a skincare regimen in which the user will first cleanse the skin before applying the composition. In this case, the skin will be stripped of most of its sebum. Clean skin with little sebum has a surface tension of about 27-28 mN/m. Therefore, some preferred compositions of the invention will have a surface tension that is greater than about 28 mN/m, preferably in the range of about 29.4 mN/m to about 33.6 mN/m. Compositions whose surface tension is in this range will wet out on sebum-poor skin, but not completely, thus slowing the rate of permeation into the skin, and reducing or eliminating irritation. In practice, we have found that preferred compositions of the invention comprise MCC at 2% to 12% by total weight of the composition. Microcrystalline cellulose is available, for example, from Dow Chemical as SunSpheres™ BIO SPF Booster.

### Active Agents

The invention may comprise one or more skin active agents. A skin active agent is one that provides a benefit to human skin, including, but not limited to improved moisturization, improved barrier function, increased skin smoothness, decreased lines and wrinkles, decreased pigmentation, whitening, and antiacne activity. Active agents may be incorporated into the aqueous phase or oil phase (if there is one). Non-limiting examples of hydrophilic (water soluble) actives include: algae extract, alpinia speciosa leaf extract, Alteromonas ferment extract, ascorbyl acid glucoside (AA2G), Citrullus lanatus (watermelon) fruit extract, crataegus monogyna (hawthorn) flower extract, hyaluronic acid, hydrolyzed yeast protein, Lactobacillus ferment, matricaria (chamomile) extract, lens esculenta (lentil) fruit extract, Paeonia suffruticosa (peony) root extract, panthenol, pyrus malus (apple) fruit extract and Saccharum officinarum extract. Non-limiting examples of hydrophobic (oil soluble) actives include Anthemis nobilis oil, bht (butylated hydroxytoluene), caffeine, Cocos nucifera (coconut) oil, salicylic acid, vitamin A, tetrahexyldecyl ascorbate and tocopheryl acetate. The concentration of all active agents combined will typically vary between 0.0001% to about 10%, by weight of the composition. While the composition is wet, the active agent is typically dispersed throughout the composition. Depending on the size of the active agent molecule, as the composition dries, at least some of the active agent settles into the pores of the polymer film, to be released slowly over time.

Of particular interest is bakuchiol. Compositions of the invention may comprise 0.1% to 10% of bakuchiol by weight of the composition, preferably 0.1% to 8% by weight of the composition, more preferably 0.5% to 6% by weight of the composition. As noted above, if the average pore size of the polymer film is about 1 μm to 5 μm, then as the composition dries, at least some bakuchiol will accumulate in the pores of the film, and be released over time. When the film contracts, as explained above, bakuchiol is urged toward the skin surface, where, with the help of microcrystalline cellulose, the contact area has been adjusted to regulate the rate of penetration of bakuchiol into the stratum corneum. With the present delivery system, the benefits of bakuchiol, such as tissue repair and cell rejuvenation, may be realized without adverse side effects. Bakuchiol is available, for example, from Syntheon Ltd., as Sytenol® A.

### Forms of the Composition

Compositions of the invention are aqueous, and may be implemented as simple mixtures or emulsions. Skin that is less hydrophobic due to sebum will benefit more from oil-in-water emulsions. The following are non-limiting examples of useful compositions of the invention.

| Ingredient | Example 1 | Example 2 | Example 3 | Example 4 |
|---|---|---|---|---|
| water | 69.56 | 70.96 | 72.35 | 73.75 |
| acrylates/vinyl | 14.91 | 11.18 | 7.46 | 3.73 |
| acetate | | | | |
| copolymer | | | | |
| propanediol | 3.20 | 2.40 | 1.60 | 0.80 |
| butylene glycol | 3.20 | 2.40 | 1.60 | 0.80 |
| sesame seed | 3.20 | 2.40 | 1.60 | 0.80 |
| oil | | | | |
| microcrystalline | 2.89 | 5.78 | 8.68 | 11.57 |
| cellulose | | | | |
| bakuchiol | 1.92 | 3.85 | 5.77 | 7.69 |
| acrylates | 0.80 | 0.60 | 0.40 | 0.20 |
| copolymer | | | | |
| preservative | 0.32 | 0.43 | 0.54 | 0.66 |
| system | | | | |

The dimensions and values disclosed herein are to be understood as modified by the term “about”. Every document cited herein, is hereby incorporated by reference in its entirety unless expressly excluded or otherwise limited. The citation of any document is not an admission that it is prior art with respect to any invention disclosed herein, or that it alone, or in any combination with any other references suggests any such invention. While particular embodiments of the present invention have been described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical aqueous composition comprising:
25%-75% of water by weight of the total composition;
0.0001% to 10%, by weight of the composition, of one or more skin active agents;
4% to 20%, by weight of the composition, of a polymer blend, wherein the polymer blend comprises a combination of acrylates/vinyl acetate copolymer and acrylates copolymer, wherein the weight ratio of acrylates/vinyl acetate copolymer to acrylates copolymer ranges from 10:1 to 30:1;
microcrystalline cellulose at 2% to 12%, by weight of the composition.

2. A topical composition according to claim 1 wherein by the one or more skin active agents includes 0.1% to 10% of bakuchiol by weight of the composition.

3. A topical composition according to claim 1 wherein the weight ratio of acrylates/vinyl acetate copolymer to acrylates copolymer ranges from 15:1 to 25:1.

4. A topical composition according to claim 1 wherein the weight ratio of acrylates/vinyl acetate copolymer to acrylates copolymer ranges from 18:1 to 22:1.

5. A topical composition according to claim 1 wherein the surface tension of the composition is greater than 28 mN/m.

6. A topical composition according to claim 5 wherein the surface tension of the composition is between about 29.4 mN/m and 33.6 mN/m.

7. A topical aqueous composition according to claim 1 wherein, upon drying the polymer blend forms a porous film wherein the average pore size of the porous film ranges from about 0.1 μm to about 10 μm.

8. A topical aqueous composition according to claim 2 wherein the average pore size of the porous film ranges from about 1 μm to about 5 μm.
